(19) United States
(12) Patent Application Publication
(10) Pub. No.: US 2023/0098198 A1
(43) Pub. Date: Mar. 30, 2023

(54) SWEAT SENSOR AND SWEAT SENSING SYSTEM

(71) Applicant: SUZHOU INSTITUTE OF NANO-TECH AND NANO-BIONICS (SINANO) , CHINESE ACADEMY OF SCIENCES, Suzhou (CN)

(72) Inventors: Shuqi WANG, Suzhou (CN); Ting ZHANG, Suzhou (CN); Mengyuan LIU, Suzhou (CN); Qifeng LU, Suzhou (CN); Xianqing YANG, Suzhou (CN); Lianhui LI, Suzhou (CN)

(73) Assignee: SUZHOU INSTITUTE OF NANO-TECH AND NANO-BIONICS (SINANO) , CHINESE ACADEMY OF SCIENCES, Suzhou (CN)

(21) Appl. No.: 17/794,990

(22) PCT Filed: Nov. 26, 2021
(86) PCT No.: PCT/CN2021/133342

§ 371 Date: Jul. 25, 2022

(30) Foreign Application Priority Data
Dec. 31, 2020 (CN) 202011631972.2

(51) Int. Cl. A61B 5/00; A61B 5/145

(52) U.S. Cl. CPC A61B 5/4266; A61B 5/14517; A61B 5/14546

(57) ABSTRACT

A sweat sensor, includes a sweat-guiding electrode layer including an insulating layer, a conductive electrode provided in the insulating layer, and a first through hole, wherein the first through hole goes through the insulating layer and the conductive electrode; an adhesive layer provided on the insulating layer, wherein the adhesive layer is provided with a second through hole communicated with the first through hole; and a water-absorbing diffusion layer provided on the adhesive layer, wherein the water-absorbing diffusion layer covers the second through hole. A sweat sensing system is further provided with a plurality of sweat sensors. The sweat sensor simultaneously and continuously detects a sweat volume and an electrolyte concentration in real time, and prevents the mixture of old and new sweat from interfering with the detection of the electrolyte concentration.

### CROSS REFERENCE TO RELATED APPLICATION

This patent application is based on and claims the priority of Chinese Patent Application No. 202011631972.2 filed on Dec. 31, 2020 and entitled “Sweat Sensor and Sweat Sensing System”.

### TECHNICAL FIELD

The present disclosure belongs to the technical field of sensors, in particular to a sweat sensor and a sweat sensing system.

### BACKGROUND ART

The abnormal changes of sweat compositions in human body during exercise are related to the blood concentration level, or can directly indicate the health condition of a human body. For example, Na⁺ is the most electrolyte in human sweat, which is the important basis of sweat secretion. The concentration of Na⁺ can reflect different kinds of symptoms of water and salt metabolism disorder in human body. For example, athletes, soldiers, workers, etc. will suffer from hypernatremia due to severe dehydration when working in extreme environments (strenuous exercise, overheated fire rescue, etc.), and the Na⁺ concentration in their sweat and blood is far higher than the normal value. If water and electrolytes are not judged and supplemented timely, it is very likely to cause serious physiological threats or even death.

At present, the traditional sweat sensor cannot simultaneously and continuously detect the sweat volume and the electrolyte concentration in real time, and cannot prevent the mixture of old and new sweat from interfering with the detection of the electrolyte concentration.

### SUMMARY

In order to solve the technical problems existing in the prior art, the present disclosure provides a sweat sensor and a sweat sensing system which simultaneously and continuously detect the sweat volume and the electrolyte concentration in real time, and can prevent the mixture of old and new sweat from interfering with the detection of the electrolyte concentration.

According to an aspect of the embodiment of the present disclosure, a sweat sensor is provided, which comprises a sweat-guiding electrode layer comprising an insulating layer, a conductive electrode provided in the insulating layer and a first through hole, wherein the first through hole goes through the insulating layer and the conductive electrode; an adhesive layer, which is provided on the insulating layer and is provided with a second through hole communicated with the first through hole; a water-absorbing diffusion layer, which is provided on the adhesive layer and covers the second through hole.

In an example of the sweat sensor provided in the above aspect, the central axis of the first through hole coincides with the central axis of the second through hole.

In an example of the sweat sensor provided in the above aspect, the conductive electrode comprises a first electrode and a second electrode, the first electrode and the second electrode are located on the same plane, and the central axis of the electrode through hole of the first electrode, the central axis of the electrode through hole of the second electrode and the central axis of the first through hole coincide with each other.

In an example of the sweat sensor provided in the above aspect, the conductive electrode comprises a first electrode and a second electrode, the first electrode and the second electrode are located on different planes, and the central axis of the electrode through hole of the first electrode, the central axis of the electrode through hole of the second electrode and the central axis of the first through hole coincide with each other.

In an example of the sweat sensor provided in the above aspect, the sweat sensor further comprises a contact layer provided on the surface of the insulating layer facing away from the adhesive layer, and the contact layer is provided with a third through hole communicated with the first through hole.

In an example of the sweat sensor provided in the above aspect, the central axis of the first through hole coincides with the central axis of the third through hole.

In an example of the sweat sensor provided in the above aspect, the insulating layer and/or the contact layer is made of polydimethylsiloxane, silicone rubber or thermoplastic polyester.

In an example of the sweat sensor provided in the above aspect, when the sweat sensor is used to detect sweat, a conductance square wave curve is obtained according to a conductance value of sweat passing through the first through hole recorded by the conductive electrode, the total sweat electrolyte concentration and the total sweat volume are simultaneously obtained through the conductance square wave curve; the amplitude of the conductance square wave curve is correlated with the real-time total sweat electrolyte concentration in the through hole, and the sweat volume and the sweat rate of sweat passing through the through hole are correlated with the time difference between conductance square waves in the conductance square wave curve.

According to another aspect of the present disclosure, a sweat sensing system is provided, which comprises a sweat-guiding electrode layer comprising an insulating layer, a plurality of conductive electrodes provided in the insulating layer and a plurality of first through holes, wherein each of the first through holes goes through the insulating layer and a corresponding one of the conductive electrodes; an adhesive layer, which is provided on the insulating layer and is provided with a plurality of second through holes, wherein the second through holes are communicated with the first through holes in one-to-one correspondence; a water-absorbing diffusion layer, which is provided on the adhesive layer and covers the plurality of second through holes.

In an example of the sweat sensing system provided in the above another aspect, each conductive electrode comprises a first electrode and a second electrode, the first electrode and the second electrode of each conductive electrode are located on different planes, the first electrodes of various conductive electrode are located on the same plane, the second electrodes of various conductive electrode are located on the same plane, and the central axis of the electrode through hole of the first electrode, the central axis of the electrode through hole of the second electrode and the central axis of the corresponding first through hole of each conductive electrode coincide with each other; the plurality of conductive electrodes are arranged in an array, the first electrodes of the conductive electrodes in the same row are connected together, and the second electrodes of the conductive electrodes in the same row are connected together.

In an example of the sweat sensing system provided in the above another aspect, the sweat sensing system further comprises a contact layer, which is provided on the surface of the insulating layer facing away from the adhesive layer, the contact layer is provided with a plurality of third through holes, and the third through holes are communicated with the first through holes in one-to-one correspondence.

Compared with the prior art, the present disclosure has the following beneficial effect.

The sweat sensor of the present disclosure can simultaneously and continuously detect the sweat volume and the electrolyte concentration in real time, and can prevent the mixture of old and new sweat from interfering with the detection of the electrolyte concentration.

### BRIEF DESCRIPTION OF THE DRAWINGS

The above and other aspects, features and advantages of the embodiments of the present disclosure will become clearer from the following description taken in conjunction with the accompanying drawings, in which:

FIG. 1 is a schematic structural diagram of a sweat sensor according to a first embodiment of the present disclosure.

FIG. 2 is a schematic diagram of the state in which a wearable device with a sweat sensor according to a first embodiment of the present disclosure is placed on the surface of human skin.

FIGS. 3A and 3B are schematic diagrams of the detection principle of a conductance square wave curve and a conductance square wave curve diagram obtained by a sweat sensor under the micro-flow injection pump test according to an embodiment of the present disclosure.

FIG. 4 is a schematic structural diagram of a sweat sensor according to a second embodiment of the present disclosure.

FIG. 5 is a schematic structural diagram of a sweat sensor according to a third embodiment of the present disclosure.

FIG. 6 is a schematic structural diagram of a sweat sensing system according to an embodiment of the present disclosure.

### DETAILED DESCRIPTION OF THE EMBODIMENTS

Hereinafter, specific embodiments of the present disclosure will be described in detail with reference to the drawings. However, the present disclosure can be implemented in many different forms, and the present disclosure should not be construed as limited to the specific embodiments set forth here. On the contrary, these embodiments are provided to explain the principles of the present disclosure and its practical application, so as to enable others skilled in the art to understand the various embodiments of the present disclosure and various modifications suitable for the specific intended application.

As used herein, the term “including” and its variants mean open terms with the meaning of “including but not limited to”. The terms such as “based on” and “according to” mean “at least partially based on” and “at least partially according to”. The terms “one embodiment” and “an embodiment” mean “at least one embodiment”. The term “another embodiment” means “at least one other embodiment”. The terms such as “first”, “second”, etc. can refer to different or identical objects. Other definitions, whether explicit or implicit, can be included below. Unless the context clearly indicates, the definition of a term is consistent throughout the specification.

FIG. 1 is a schematic structural diagram of a sweat sensor according to a first embodiment of the present disclosure. In FIG. 1, the diagram (B) shows a top view of a sweat sensor according to a first embodiment of the present disclosure. It is noted that in the diagram (B), in order to clearly show the electrode structure, the adhesive layer 3 and the water-absorbing diffusion layer 4 are not shown. The diagram (A) shows a cross-sectional view of a sweat sensor according to a first embodiment of the present disclosure taken along the line a-a′ in the diagram (B). Of course, the diagram (A) additionally shows a human skin system.

As shown in FIG. 1, a sweat sensor according to a first embodiment of the present disclosure comprises a sweat-guiding electrode layer 2, an adhesive layer 3, and a water-absorbing diffusion layer 4.

Specifically, the sweat-guiding electrode layer 2 comprises an insulating layer 21, a conductive electrode (not labeled) provided in the insulating layer 21, and a first through hole (not labeled), wherein the first through hole goes through the insulating layer 21 and the conductive electrode. In one example, the conductive electrode comprises a first electrode 221 and a second electrode 222, wherein the first electrode 221 and the second electrode 222 are located on the same plane, and the central axis of the electrode through hole (not labeled) of the first electrode 221, the central axis of the electrode through hole (not labeled) of the second electrode 222 and the central axis of the first through hole coincide with each other.

In one example, the insulating layer 21 is mainly made of a flexible insulating polymer material, which may be polydimethylsiloxane, silicone rubber, thermoplastic polyester, etc. The thickness of the insulating layer 21 is between 0.1 mm and 2 mm.

The first electrode 221 and the second electrode 222 are embedded inside the insulating layer 21 and located at the middle part in the thickness direction. The first electrode 221 and the second electrode 222 can be thin-film electrodes with certain thickness and width made of carbon nanotubes, graphene, carbon black, carbon fiber, etc., or thin-film electrodes with certain thickness and width made of other materials such as conductive testing metals such as gold, platinum, copper, etc. The thickness of the first electrode 221 and the second electrode 222 is between 0.01 mm and 1 mm, and the width (line width) of the first electrode 221 and the second electrode 222 is smaller than the diameter of each through hole (e.g., an electrode through hole, a first through hole, etc.).

Here, the embedding method of the first electrode 221 and the second electrode 222 in the insulating layer 21 is not particularly limited. For example, in one example, first, an insulating layer 21 is prepared, and the first electrode 221 and the second electrode 222 located on the same level are prepared on the insulating layer 21 using a method such as screen printing. Finally, another insulating layer 21 is prepared on the insulating layer 21, the first electrode 221 and the second electrode 222, and the first electrode 221 and the second electrode 222 are embedded in the position inside the insulating layer 21. In another example, first, an electrode-shaped mold template is prepared using a machining method. The prepolymer, of which the insulating layer 21 is made, is then poured into the mold template using a template replication method, and after the prepolymer is peeled off after being cured and molded, a groove with an electrode shape is formed. The first electrode 221 and the second electrode 222 are filled and prepared in the groove, and then another insulating layer 21 is prepared on the first electrode 221 and the second electrode 222. Finally, the first electrode 221 and the second electrode 222 are embedded in the position inside the insulating layer 21.

At the middle of the insulating layer 21, the first electrode 221 and the second electrode 222, a through hole 23 (consisting of the first through hole, the electrode through hole, etc.) is prepared by a laser cutting method, a template method or a mechanical punching method, and the diameter of the through hole 23 is between 0.5 mm and 2 mm. The effective test surfaces of the first electrode 221 and the second electrode 222 are exposed to the inner wall surface of the through hole 23. Preferably, the cylindrical inner wall of the through hole 23 and the surfaces of the first electrode 221 and the second electrode 222 exposed to the through hole show hydrophobic properties. Therefore, the hydrophobic materials can be selected according to the properties of the materials of the insulating layer 21 and the electrode, and the hydrophobic through hole can also be realized by post-treatment methods, such as silane reagent treatment.

The adhesive layer 3 is provided on the insulating layer 21, and the adhesive layer 3 is provided with a second through hole (not shown) communicated with the first through hole. That is, the through hole 23 goes through the insulating layer 21, the adhesive layer 3, the first electrode 221 and the second electrode 222. The part of the through hole 23 in the insulating layer 21 is set as the first through hole, the part of the through hole 23 in the adhesive layer 3 is set as the second through hole, the part of the through hole 23 in the first electrode 221 is set as the electrode through hole of the first electrode 221, and the part of the through hole 23 in the second electrode 222 is set as the electrode through hole of the second electrode 222.

The adhesive layer 3 is a viscous film fixedly connected between the sweat-guiding electrode layer 2 and the water-absorbing diffusion layer 4, and comprises an ultra-thin double-sided adhesive tape with a fixed thickness (0.01 mm to 0.05 mm in thickness), a prepolymer of viscoelastic polymer, etc. In one example, the adhesive layer 3 prepares a second through hole with the same size as the first through hole at the overlapping position of the first through hole of the sweat-guiding electrode layer 2 by a laser cutting method, a template method or a mechanical punching method, so that sweat flows through the first through hole and the second through hole. Further, the central axis of the first through hole coincides with the central axis of the second through hole.

The water-absorbing diffusion layer 4 is provided on the adhesive layer 3 and covers the second through hole. In one example, the water-absorbing diffusion layer 4 is a film made of a hydrophilic material, including but not limited to water-absorbing materials such as clothing fabrics, paper-based cellulosic films, gels and the like. In this embodiment, the water-absorbing diffusion layer 4 can take clothing itself as the water-absorbing layer, and preferably take breathable sweat-permeable sports tights, wrist guards, palm guards, elbow guards, sweat-absorbing belts and the like as the water-absorbing layer. The thickness of the water-absorbing diffusion layer 4 is not limited. The water-absorbing diffusion layer 4 is integrated with the sweat-guiding electrode layer 2 through the adhesive layer 3 to form a sweat sensor.

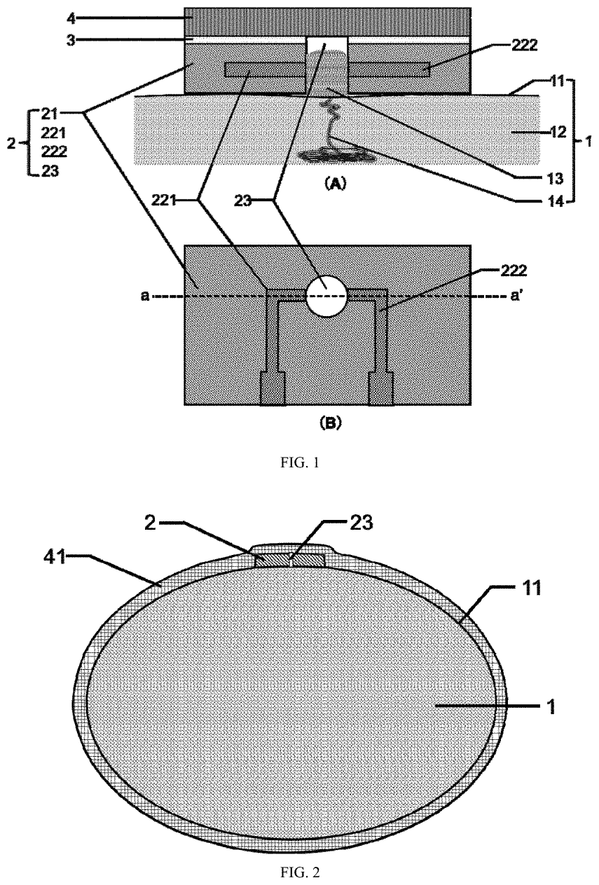

FIG. 2 is a schematic diagram of the state in which a wearable device with a sweat sensor according to a first embodiment of the present disclosure is placed on the surface of human skin. As shown in FIG. 2, a sweat sensor according to the first embodiment of the present disclosure is wrapped and fixed by a belt made of elastic fabrics and elastic materials to form a wearable device. Furthermore, the wearable device can be integrated and compatible with sports tights, wrist guards, palm guards, elbow guards, sweat-absorbent belts and other fabrics to form an elastic water-absorbing fixing belt device 41.

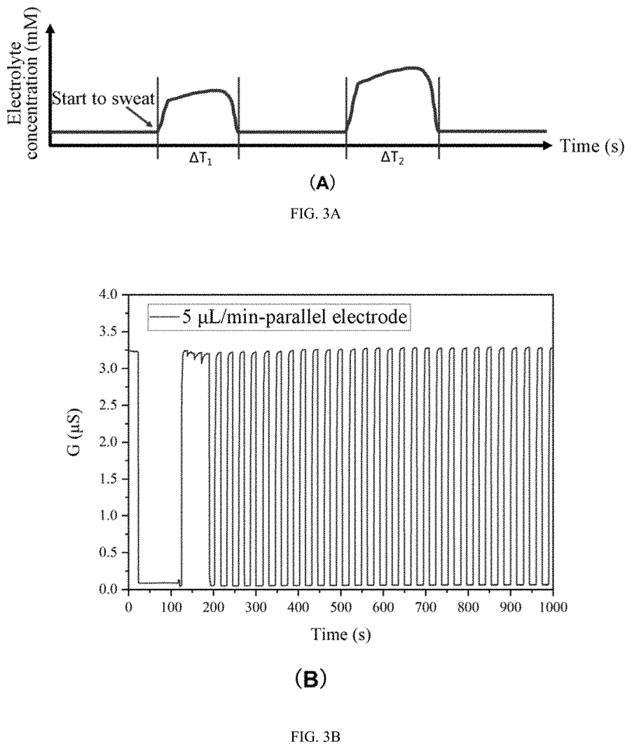

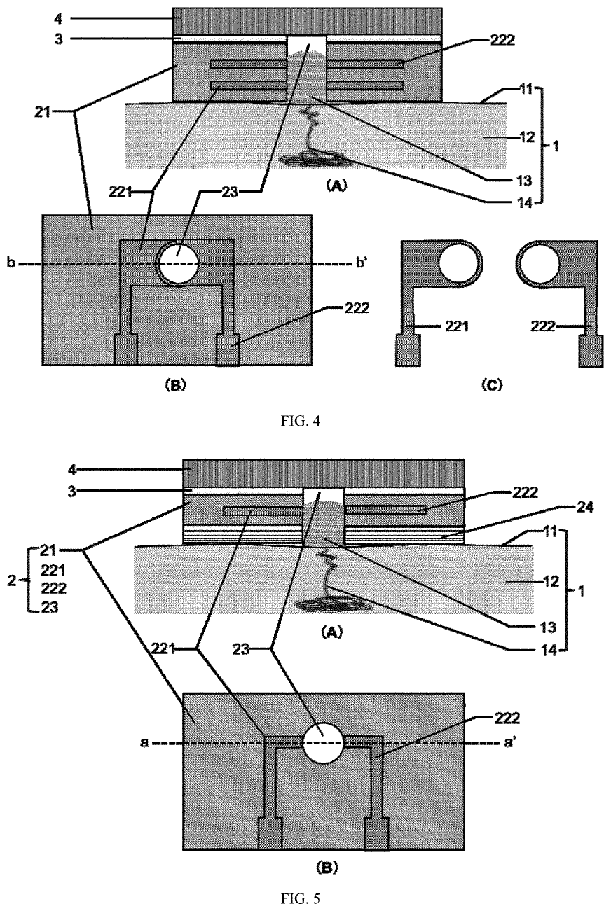

When the sweat sensor according to the embodiment of the present disclosure is provided on the human skin 1, sweat 13 is secreted by sweat glands 14 in the hypodermis 12. When being secreted from sweat glands 14, sweat 13 has a certain pressure up to 70,000 Nm⁻², which is enough to pump sweat 14 into the through hole 23 to be quickly absorbed by the water-absorbing diffusion layer 4. When sweat 13 passes through the inner wall of the through hole 23, the parallel electrodes (i.e., the first electrode 221 and the second electrode 222) exposed to the through hole 23 will record the conductance value of the sweat liquid or the sweat droplet in real time. FIG. 3A is a schematic diagram of the detection principle of a conductance square wave curve, where ΔT₁ represents the duration of a first conductance square wave and ΔT₂ represents the duration of a second conductance square wave. FIG. 3B is a conductance square wave curve diagram obtained by a sweat sensor under the micro-flow injection pump test according to an embodiment of the present disclosure.

As shown in FIG. 3B, the height or amplitude of the conductance square wave curve is correlated with the real-time total sweat electrolyte concentration in the through hole 23, and the sweat volume and sweat rate of sweat droplets passing through the through hole 23 are correlated with the time difference between the conductance square wave curve and the next conductance square wave curve. Therefore, the sweat sensor according to the embodiment of the present disclosure can successfully distinguish the sweat electrolyte concentration from the sweat volume through a real-time continuous conductance square wave curve. At the same time, the sweat sensor has the advantage that the accuracy is not interfered by the mixture of old and new sweat.

FIG. 4 is a schematic structural diagram of a sweat sensor according to a second embodiment of the present disclosure. In FIG. 4, the diagram (B) shows a top view of a sweat sensor according to a second embodiment of the present disclosure. It is noted that in the diagram (B), in order to clearly show the electrode structure, the adhesive layer 3 and the water-absorbing diffusion layer 4 are not shown. The diagram (A) shows a cross-sectional view of a sweat sensor according to a second embodiment of the present disclosure taken along the line a-a′ in the diagram (B). Of course, the diagram (A) additionally shows a human skin system. The diagram (C) shows a schematic structural diagram of the first electrode and the second electrode in the sweat sensor according to the second embodiment of the present disclosure.

As shown in FIG. 4, the structure here is different from the structure of the sweat sensor of the first embodiment shown in FIG. 1 in that the first electrode 221 and the second electrode 222 are not located on the same plane. For example, the second electrode 222 is above the first electrode 221, so that the electrode through hole of the second electrode 222 overlaps with the electrode through hole of the first electrode 221 from top to bottom.

FIG. 5 is a schematic structural diagram of a sweat sensor according to a third embodiment of the present disclosure. In FIG. 5, the diagram (B) shows a top view of a sweat sensor according to a third embodiment of the present disclosure. It is noted that in the diagram (B), in order to clearly show the electrode structure, the adhesive layer 3 and the water-absorbing diffusion layer 4 are not shown. The diagram (A) shows a cross-sectional view of a sweat sensor according to a third embodiment of the present disclosure taken along the line a-a′ in the diagram (B). Of course, the diagram (A) additionally shows a human skin system.

As shown in FIG. 5, the structure here is different from the structure of the sweat sensor of the first embodiment shown in FIG. 1 in that the sweat sensor according to the third embodiment of the present disclosure further comprises a contact layer 24, which is provided on the surface of the insulating layer 21 facing away from the adhesive layer 3. The contact layer 24 is provided with a third through hole (not shown) communicated with the first through hole. Further, the central axis of the first through hole coincides with the central axis of the third through hole, that is, the through hole 23 goes through the contact layer 24.

The contact layer 24 has two functions: first, it is convenient to adjust the thickness of the sweat-guiding electrode layer 2; second, when the hardness of the insulating layer 21 is not suitable for direct contact with the skin, the contact layer 24 can achieve good contact and attachment with the skin. The material of the contact layer 24 includes, but is not limited to, polydimethylsiloxane, silicone rubber, thermoplastic polyester, etc. The close bonding between the contact layer 24 and the insulating layer 21 can be achieved by common techniques such as cross-linking and bonding.

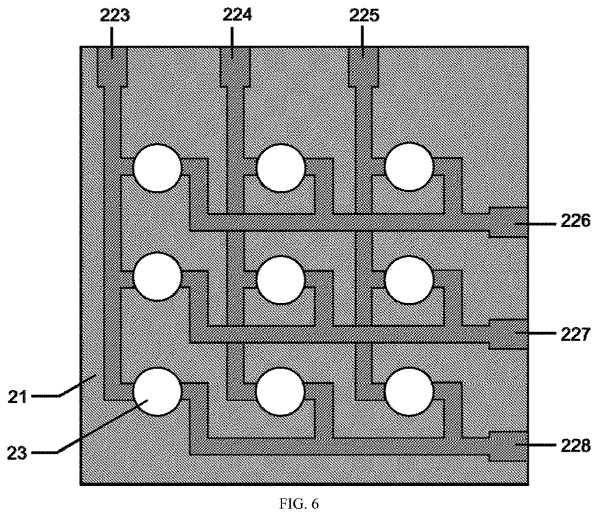

FIG. 6 is a schematic structural diagram of a sweat sensing system according to an embodiment of the present disclosure. For the convenience of description, only conductive electrodes and insulating layers are shown in FIG. 6.

As shown in FIG. 6, the sweat sensing system according to the embodiment of the present disclosure comprises a plurality of sweat sensors according to the second embodiment of the present disclosure shown in FIG. 4. The plurality of sweat sensors are arranged in an array.

In this case, the insulation layer 21, the adhesive layer 3, the water-absorbing diffusion layer 4 and/or the contact layer 24 (if provided) of each sweat sensor are integrated. That is, a plurality of conductive electrodes are provided in an insulating layer 21. An adhesive layer 3 and a water-absorbing diffusion layer 4 are laminated on the insulating layer 21 at a time, and a contact layer 24 is provided on the surface of an insulating layer 21 facing away from an adhesive layer 3.

Of course, each through hole 23 also goes through an adhesive layer 3 to form a plurality of second through holes, and also goes through a contact layer 24 to form a plurality of third through holes. That is, the first through hole (including each electrode through hole), the second through hole and the third through hole are communicated with each other in one-to-one correspondence.

When the plurality of sweat sensors are distributed in an array, the first electrodes of various conductive electrode are located on the same plane, the second electrodes of various conductive electrode are located on the same plane, and the first electrodes and the second electrodes are located on different planes. Therefore, all the first electrodes of the conductive electrodes of each column are connected together and connected to the column conductive terminals, while all the second electrodes of the conductive electrodes of each row are connected together and connected to the row conductive terminals. For example, in FIG. 6, all the first electrodes of a first column of conductive electrodes are connected together and connected to the column conductive terminal 223, all the first electrodes of a second column of conductive electrodes are connected together and connected to the column conductive terminal 224, and all the first electrodes of a third column of conductive electrodes are connected together and connected to the column conductive terminal 225. All second electrodes of a first row of conductive electrodes are connected together and connected to the row conductive terminal 226, all second electrodes of a second row of conductive electrodes are connected together and connected to the row conductive terminal 227, and all second electrodes of a third row of conductive electrodes are connected together and connected to the row conductive terminal 228.

With the sweat sensing system provided above, the data of a plurality of sampling points can be obtained by arranging a plurality of conductive electrodes in an array, so that the accuracy of analysis of sweat volume per unit area and sweat electrolyte concentration can be further improved.

The specific embodiments of the present disclosure have been described above. Other embodiments are within the scope of the appended claims.

The terms “exemplary” and “example” used throughout this specification mean “serving as an example, instance or illustration”, rather than mean “preferable to” or “advantageous over” other embodiments. For the purpose of providing an understanding of the described technology, the detailed description comprises specific details. However, these techniques may be practiced without these specific details. In some instances, in order to avoid obscuring the concepts of the described embodiments, well-known structures and devices are shown in the form of a block diagram.

The alternative implementation of the embodiments of the present disclosure is described in detail with reference to the drawings hereinabove, but the embodiments of the present disclosure are not limited to the specific details of the above implementation. Within the technical concept of the embodiments of the present disclosure, many simple modifications can be made to the technical solutions of the embodiments of the present disclosure, and these simple modifications all belong to the protection scope of the embodiments of the present disclosure.

The above description of this specification is provided to enable those skilled in the art to make or use this specification. Various modifications made to this specification are obvious to those skilled in the art, and the general principles defined herein can also be applied to other modifications without departing from the scope of protection of this specification. Therefore, this specification is not limited to the examples and designs described herein, but is in agreement with the widest scope consistent with the principles and novel features disclosed herein.

1. A sweat sensor, comprising:
a sweat-guiding electrode layer comprising an insulating layer, a conductive electrode provided in the insulating layer, and a first through hole, wherein the first through hole goes through the insulating layer and the conductive electrode;
an adhesive layer provided on the insulating layer, wherein the adhesive layer is provided with a second through hole communicated with the first through hole; and
a water-absorbing diffusion layer provided on the adhesive layer, wherein the water-absorbing diffusion layer covers the second through hole.

2. The sweat sensor according to claim 1, wherein a central axis of the first through hole coincides with a central axis of the second through hole.

3. The sweat sensor according to claim 2, wherein the conductive electrode comprises a first electrode and a second electrode, wherein the first electrode and the second electrode are located on a same plane, and a central axis of an electrode through hole of the first electrode, a central axis of an electrode through hole of the second electrode, and the central axis of the first through hole coincide with each other.

4. The sweat sensor according to claim 2, wherein the conductive electrode comprises a first electrode and a second electrode, wherein the first electrode and the second electrode are located on different planes, and a central axis of an electrode through hole of the first electrode, a central axis of an electrode through hole of the second electrode, and the central axis of the first through hole coincide with each other.

5. The sweat sensor according to claim 1, further comprising a contact layer provided on a surface of the insulating layer facing away from the adhesive layer, wherein the contact layer is provided with a third through hole communicated with the first through hole.

6. The sweat sensor according to claim 5, wherein a central axis of the first through hole coincides with a central axis of the third through hole.

7. The sweat sensor according to claim 1, wherein when the sweat sensor is used to detect sweat, a conductance square wave curve is obtained according to a conductance value of sweat passing through the first through hole recorded by the conductive electrode, wherein a real-time total sweat electrolyte concentration and a total sweat volume are simultaneously obtained through the conductance square wave curve; and wherein an amplitude of the conductance square wave curve is correlated with the real-time total sweat electrolyte concentration in the first through hole, and the total sweat volume and a sweat rate of sweat passing through the first through hole are correlated with a time difference between conductance square waves in the conductance square wave curve.

8. A sweat sensing system, comprising:
a sweat-guiding electrode layer comprising an insulating layer, a plurality of conductive electrodes provided in the insulating layer, and a plurality of first through holes, wherein each of the plurality of first through holes goes through the insulating layer and a corresponding one of the conductive electrodes;
an adhesive layer provided on the insulating layer, wherein the adhesive layer is provided with a plurality of second through holes, and the plurality of second through holes are communicated with the plurality of first through holes in one-to-one correspondence; and
a water-absorbing diffusion layer provided on the adhesive layer, wherein the water-absorbing diffusion layer covers the plurality of second through holes.

9. The sweat sensing system according to claim 8, wherein each conductive electrode of the plurality of conductive electrodes comprises a first electrode and a second electrode, wherein the first electrode and the second electrode of each conductive electrode are located on different planes, and wherein the first electrodes of the plurality of conductive electrodes are located on a first plane, the second electrodes of the plurality of conductive electrodes are located on a second plane, and a central axis of an electrode through hole of the first electrode, a central axis of an electrode through hole of the second electrode, and a central axis of the corresponding first through hole of each conductive electrode coincide with each other; and
the plurality of conductive electrodes are arranged in an array, wherein the first electrodes of the plurality of conductive electrodes in a same row are connected together, and the second electrodes of the plurality of conductive electrodes in a same row are connected together.

10. The sweat sensing system according to claim 8, further comprising a contact layer provided on a surface of the insulating layer facing away from the adhesive layer, wherein the contact layer is provided with a plurality of third through holes, and the plurality of third through holes are communicated with the plurality of first through holes in one-to-one correspondence.

11. The sweat sensing system according to claim 9, further comprising a contact layer provided on a surface of the insulating layer facing away from the adhesive layer, wherein the contact layer is provided with a plurality of third through holes, and the plurality of third through holes are communicated with the plurality of first through holes in one-to-one correspondence.
